(19) United States
(12) Patent Application Publication
(10) Pub. No.: US 2009/0062771 A1
(43) Pub. Date: Mar. 5, 2009

(54) BALLOON-TIPPED NASOGASTRIC FEEDING TUBE

(76) Inventors: Nicholas A. Tarola, Aldan, PA (US); Evelio Rodriguez, Cherry Hill, NJ (US)

Correspondence Address: DAVID S. RESNICK, NIXON PEABODY LLP, 100 SUMMER STREET, BOSTON, MA, 02110-2131, US

(21) Appl. No.: 12/160,148

(22) PCT Filed: Jan. 3, 2007
(86) PCT No.: PCT/US07/00170

§ 371 Date: Jul. 7, 2008

Related U.S. Application Data
(60) Provisional application No. 60/756,803, filed on Jan. 5, 2006.

(51) Int. Cl. A61M 39/00

(52) U.S. Cl. 604/516; 604/103.03

(57) ABSTRACT

A feeding tube for insertion into a digestive system of a subject includes a tube having a distal end and a proximal end. The tube defines a lumen extending from the proximal end to the distal end. The lumen has an inner diameter sized to accommodate the passage of alimentation to the digestive system of the subject. The distal end is arranged to be positioned through the subject's esophagus and into the digestive system of the subject. An expandable member is disposed at the distal end. The expandable member is expandable to an outer diameter that is larger than an inner diameter of the subject's respiratory tract to prevent insertion of said lumen therein.

### BACKGROUND OF THE INVENTION

1. Field of the Invention

The present invention relates to a naso-gastric or naso-gastric-duodenal feeding tube device and more particularly to a feeding tube or catheter having an inflatable tip for proper placement of the device only within the patient's digestive tract.

2. Description of the Related Art

Nasal and oral inserted feeding tubes are used for the short term feeding (30 days or less) of patients requiring nutritional support. In practice, the tube is inserted either into the mouth or nose of the patient through the patient's pharynx, through the esophagus and into the stomach or small intestine. A major complication of this process is the potential of passing the feeding tube into the trachea, and then deeper into the respiratory tract. Conventional plastic feeding tubes have a fairly stiff tip that can easily puncture lung parenchyma if advanced too far into the airway, ending up in the pleural space causing a pneumothorax. This can cause damage to the respiratory tract, and in serious cases, the death of the patient.

The above complication is often seen in the sedated or intubated patient who is unable to give an appropriate response, such as a grimace from pain or coughing from passage into the trachea.

There are known methods used to confirm proper placement of feeding tubes in the esophagus. These current methods include fluoroscopy, chest X-rays and specially adapted stethoscopes. Fluoroscopy is time consuming, extremely expensive, and expose the patient and medical staff to high doses of radiation. Additionally, specially tipped feeding tubes used along with an external locator device have also been proposed in the past. These specially adapted stethoscopes are difficult to use because of the need to differentiate sounds, especially in noisy hospital environments. The specially tipped feeding tubes and corresponding external locator device systems are also very expensive and require extensive knowledge of anatomical landmarks. As these tubes are advanced, the external locator detects and indicates the tube's position. However, tracheal intubation cannot be ruled out until it is determined by the external locator readout that the tube is past the lungs. However, this may occur at too late of a time to prevent damage, accordingly making this is an ineffectual method of monitoring and confirming feeding tube placement. See U.S. Pat. No. 5,037,387.

Traditional feeding tubes include an upper end with ports for delivering nutrients. The opposed end is insertable into a patient and nutrient is delivered therethrough. Such a nasogastric dob-hoff feeding tube is manufactured by Viasys MedSystems of Wheeling, Il. However, the insertable end of such a tube can be accidentally inserted in the patient's respiratory tract.

Non-feeding tube type catheters are known to use an inflatable tip. For example, the Swan Ganz pulmonary artery catheter, as manufactured by Edwards Lifesciences of Irvine, Calif., has a balloon at the distal end for the purpose of measuring pulmonary arterial pressure. The balloon is also used to occlude the blood vessel such that the flow of blood propels the catheter forward. However, the Swan Ganz catheter is inappropriate for use as an enteral feeding tube because its bore size is too small for the passage of alimentation, as well as the placement of the distal port at the far distal end of the tube.

U.S. Pat. No. 6,582,395 discloses a foley type feeding tube having a balloon bolster disposed on an end thereof that is inflated after the tube end is positioned within the patient's stomach. However, the balloon bolster is not designed to be inflated prior to reaching the stomach as a guide or as a measure to prevent misinsertion.

Thus, there is a need in the art for a feeding tube device that prevents distal airway intubation during insertion, particularly in patients who are sedated or intubated.

### SUMMARY OF THE INVENTION

One aspect of the present invention is to provide a nasogastric feeding tube for insertion solely into a digestive system of a subject, and not the respiratory tract.

Another aspect of the present invention is to provide a feeding tube having an inflatable balloon at a tip thereof that can prevent the tip of the feeding tube from puncturing the parenchyma of the lung if positioned incorrectly.

Still yet another aspect of the present invention is to provide a feeding tube that can be inserted in an unconscious or supine patient with limited risk of mis-insertion into the patient's distal respiratory tract.

According to the aspects of the present invention a feeding tube for insertion into a digestive system of a subject includes a tube having a distal end and a proximal end. The tube defines a lumen extending from the proximal end to the distal end. The lumen has an inner diameter sized to accommodate the passage of alimentation to the digestive system of the subject. The distal end is arranged to be positioned through the subject's esophagus and into the digestive system of the subject. An expandable member is disposed at the distal end. The expandable member is expandable to an outer diameter that is larger than an inner diameter of the subject's distal respiratory tract to prevent insertion of said lumen therein.

In one embodiment, the balloon is inflatable to a volume of about 4.0 cc to about 8.0 cc for the adult patient. In one embodiment, the balloon is inflated via an inflation port at the proximal end external to the subject. In one embodiment, the inflation of the balloon is mirrored by the inflation of a second balloon at the proximal end.

According to another aspect of the present invention there is provided a method for providing fluidic communication with a digestive system of a subject comprising the steps of providing a feeding tube. The tube has a distal end and a proximal end. A lumen extends from the proximal end to the distal end. The lumen has an inner diameter sized to accommodate the passage of alimentation to the digestive system of the subject. The distal end is arranged to be positioned through the subject's esophagus and into the digestive system of the subject. An expandable member is disposed at the distal end. The member is expandable to an outer diameter that is larger than an inner diameter of the subject's distal respiratory tract to prevent insertion of the lumen therein. The distal end is inserted into the subject and the expanding member inflated. Thereafter the distal end is steered through the esophagus into the digestive system, wherein said expanding member prevents insertion of the distal end into a bronchial of the respiratory tract of the subject.

### BRIEF DESCRIPTION OF THE DRAWINGS

FIG. 1 illustrates a feeding tube of the present invention inserted into a patient.

FIG. 2 is a perspective view of the feeding tube of the present invention with the proximal and distal ends shown enlarged.

FIG. 3 is a cross-sectional view of the upper portion of the feeding catheter of the present invention.

FIG. 4 illustrates how the feeding tube of the present invention cannot be mis-inserted into the distal respiratory tract of the subject.

### DETAILED DESCRIPTION OF THE PREFERRED EMBODIMENTS

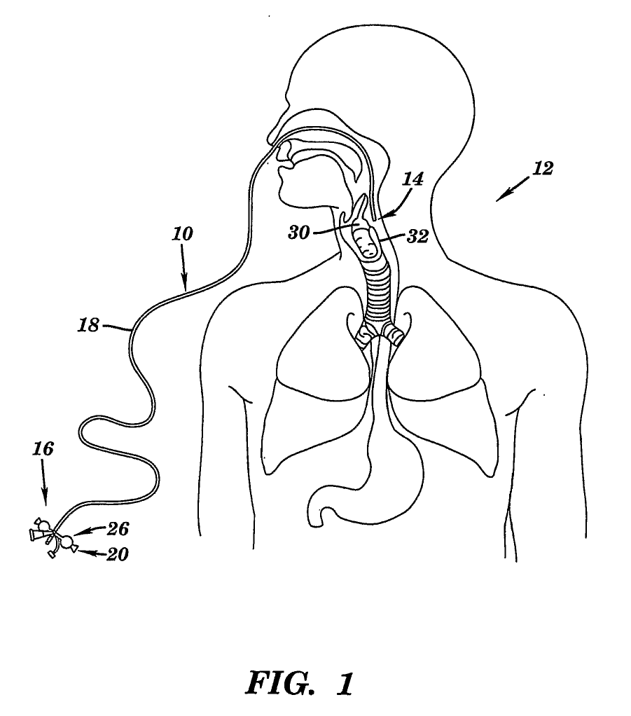

As shown in FIG. 1, the feeding tube 10 in accordance with the present invention is shown inserted into a patient 12. Tube 10 defines a lumen 18 and has a distal end 14 and a proximal end 16. As will be fully described herein, tube 10 prevents improper placement of the tube in the distal airway of the patient, thereby preventing injury or the like resulting from improper placement. Tube 10 is flexible and made of known materials such as medical grade silicon or polyurethane. Although, other materials are contemplated by the present invention. Proximal end 16 includes a feeding port 17 that communicates with lumen 18 and is capable of delivering nutrients as is known.

The lumen 18 has an internal diameter that is large enough to provide alimentation to the digestive system of the subject, and has a bore size from about 8 to about 16 french for adult use. In one embodiment, the bore size is 10 french for use with adults. For pediatric use, the bore size is about 6 to about 8 french. It should be appreciated that other diameters or bore sizes of the lumen are contemplated by the present invention. For example, for use in pediatric or smaller patients a bore size in a lower range can be used.

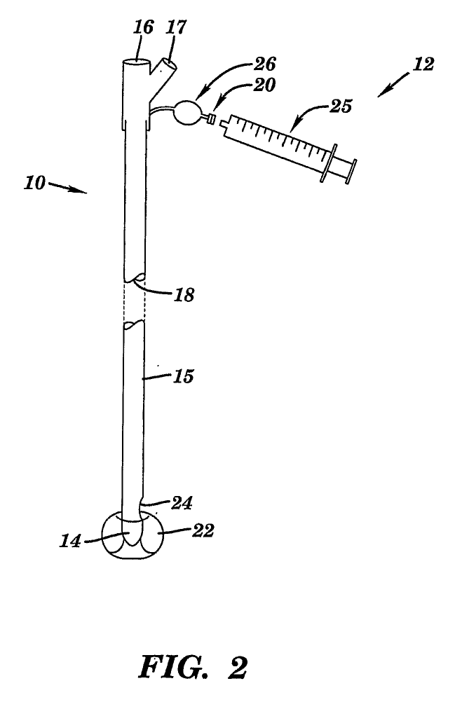

Referring to FIG. 2, distal end 14 includes an expandable member 22. Member 22 can be a balloon 22 disposed about the tip of the tube. It should be appreciated that the present invention contemplates other forms of expanding members, for example, movable arms or umbrellas that can be remotely manipulated fluidically or by other mechanical means

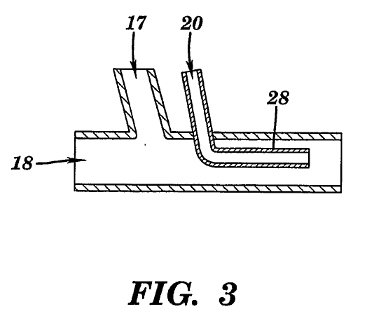

To expand member 22 proximal end 16 includes a supplementary port 20. In the embodiment of FIG. 2, balloon 22 is positioned at the far distal end 14 of tube 10. Port 20 communicates with balloon 22 to inflate the same. As shown in FIG. 3 the inflation of balloon 22 is conducted by way of a separate inflation tube 28 inside the lumen 18 of tube 10.

In one embodiment, the lumen of inflation tube 28 is 0.4 mm in diameter. Fluid, such as water or air, is delivered to port 20 via a syringe 25 or other means. The syringe 25 may connect to the port 20 by means of a luer lock. By sequential delivery of the fluid the expansion of the balloon can be controlled remotely. This graduated expansion can allow the device to be used in different sized patients.

Inflation of the balloon 22 may be monitored via a cuff 26, which is positioned in communication with the balloon inflation port 20. The cuff 26 may be positioned in the vicinity of inflation port 20 at proximal end 16. As will be discussed further herein, preferably balloon 22 is not inflated to its full diameter until inserted into the oropharynx.

The balloon has a diameter, at full inflation, of less than 2 cm. The average diameter of the esophagus in an adult is approximately 2 cm and the average diameter of the trachea of an adult is approximately 1.4-2.7 cm (Ann R Coll Surg Engl 1984, 66:356-358). The average adult bronchial diameter is approximately 0.9 to 1.6 cm (J Cardiothorac Vasc Anesth. 1995 April; 9(2):119-21.) Therefore, when inflated balloon 22 has a diameter that is too large to enter the bronchial passages. In one preferred embodiment, the balloon has a diameter, at full inflation, of more than 1 cm. In one embodiment, the balloon has an inflation volume of about 4.0 to about 8.0 cc. For use with pediatric subjects, the skilled artisan may readily adapt the balloon inflation size to a more appropriate diameter. In one embodiment, for use with pediatric subjects, the balloon has an inflation volume of about 0.5 to about 2.0 cc. In another embodiment, for use with pediatric subjects, the balloon has an inflation volume of about 2.0 to 4.0 cc.

As shown in FIG. 2, distal end 14 includes a bore 24 that communicates with tube 18. Bore 24 may be located 1 cm from distal end 14. Alternatively, multiple bores 24 are included at distal end 14. Bore 24 is free of balloon 22 when balloon 22 is deflated such that the fluids and nutrients can be delivered to the patient.

During use, the user inserts the proximal end 14 of the tube 10 through the patient's nose or mouth as shown in FIG. 1 for passage of the end 14 through the subject's pharynx. The goal is to prevent passage of the distal end 14 into the distal airway, rather than through the esophagus 32. To this end, during the insertion process, subsequent to passage into the pharynx, the balloon 22 is inflated via the balloon inflation port 20. Balloon inflation preferably Occurs at about 7 to about 12 cm from the entrance of the nares in the oropharynx.

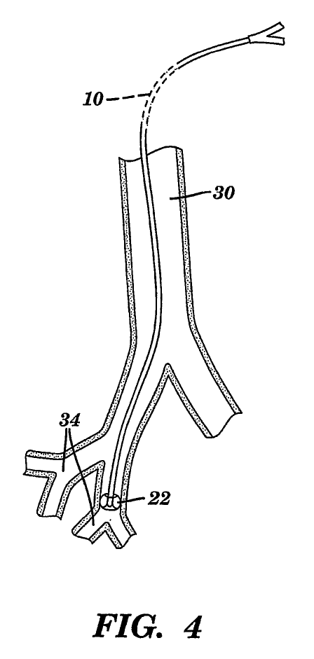

As discussed supra, in the event that the distal end 14 is inserted into the trachea 30 by accident, the balloon 22 serves two purposes. First, the large diameter of the balloon 22 prevents insertion of the tube 10 beyond the primary bronchial airway 34 of the patient, as shown in FIG. 4. Secondly, the rounded surface of the balloon 22 prevents damage to the airways of the patient. If insertion is blocked by the balloon 22, then insertion into the airway is suggested. The balloon may be deflated and the tube may then be partially withdrawn to the oropharynx, the balloon is reinflated and the tube may be reinserted until the end 14 passes behind the trachea and into the subject's esophagus. In such orientation, the balloon 22 will not block passage of the tube into the digestive system of the subject.

Once the tube 10 has been properly placed with distal end 14 in the patient's esophagus and in communication with the patient's stomach, the tube can remain in the stomach or, alternatively, the tube may be advanced to the small intestine. This involves deflating the balloon 22. The gastroesophageal junction is located about 41 cm from the patient's incisors. After 45-50 cm of the tube is inserted into the patient, the balloon 22 may be deflated. In smaller patients, e.g., pediatric patients, the length of tube inserted is accordingly shorter. At this point, the patient may be fed by the usual technique of passing liquid food through the proximal end of the tube for delivery into the patient's small intestine. Radiographic confirmation of proper placement may be performed prior to passing liquid through the tube for delivery into the patient's digestive system.

The tube 10 may be further modified with the addition of a weight at distal end 14 for use in propelling the device by peristaltic action into the small intestines of the subject. A radio-opaque stripe and distance marks 15 can further be provided on the tube for accurate locating of the tube in the subject. The tube 10 may have a double lumen for the purpose of withdrawing stomach contents as well as instilling fluid into the stomach.

Although the present invention has been described in relation to particular embodiments thereof, many other variations and modifications and other uses will become apparent to those skilled in the art. It is preferred therefore, that the present invention be limited not by the specific disclosure herein, but only by the appended claims.

What is claimed is:

1. A feeding tube for insertion into a digestive system of a subject, comprising:
a tube having a distal end and a proximal end, said tube defining a lumen extending from said proximal end to said distal end, said lumen having an inner diameter sized to accommodate the passage of alimentation to the digestive system of the subject, wherein said distal end is arranged to be positioned through the subject's esophagus and into the digestive system of the subject, and
an expandable member disposed at said distal end, said member being expandable to an outer diameter that is larger than an inner diameter of the subject's distal respiratory tract to prevent insertion of said lumen therein.

2. The feeding tube of claim 1, wherein the expandable member comprises a balloon that is inflatable to a diameter of about 4.0 to about 8.0 cc.

3. The feeding tube of claim 1, wherein the subject is a pediatric subject and the expandable member comprises a balloon that is inflatable to a diameter of about 0.5 to about 4.0 cc.

4. The feeding tube of claim 1, wherein said tube includes radio-opaque markings.

5. The feeding tube of claim 1, further comprising an inflation port disposed at the proximal end of said tube external to the subject for inflating said balloon.

6. The feeding tube of claim 6, further comprising an additional inflation port disposed at said proximal end for inflating said balloon.

7. The feeding tube of claim 1, wherein said tube is flexible.

8. A method for providing fluidic communication with a digestive system of a subject comprising the steps of:
providing a nasogastric feeding tube, said tube having a distal end and a proximal end, and defining a lumen extending from said proximal end to said distal end, said lumen having an inner diameter sized to accommodate the passage of alimentation to the digestive system of the subject, wherein said distal end is arranged to be positioned through the subject's esophagus and into the digestive system of the subject, and an expandable member disposed at said distal end, said member being expandable to an outer diameter that is larger than an inner diameter of the subject's respiratory tract to prevent insertion of said lumen therein;
inserting said distal end into the subject;
inflating the expanding member; and
steering the distal end through the esophagus into the digestive system, wherein said expanding member prevents insertion of the distal end into a bronchial of the respiratory tract of the subject.

9. The method of claim 8, further comprising the step of retracting insertion of said distal end if a blockage of said expanding member is noted.

10. The method of claim 8, wherein the expanding member is inflated at about 7 to about 12 cm from the subject's nares in the subject's oropharynx.
